(19) United States
(12) Patent Application Publication
(10) Pub. No.: US 2010/0069709 A1
(43) Pub. Date: Mar. 18, 2010

(54) APPARATUS AND METHOD FOR REINFORCEMENT OF PENILE CYLINDERS

(75) Inventor: Randy L. Morningstar, Brooklyn Park, MN (US)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/561,472

(22) Filed: Sep. 17, 2009

(30) Foreign Application Priority Data
Sep. 18, 2008 (DK) PA 2008 01306

(51) Int. Cl. A61F 5/00

(52) U.S. Cl. 600/40

(57) ABSTRACT

An apparatus for reinforcement of a penile cylinder includes a fixture having a vertical axis, a horizontal axis, a cylinder tip cavity that is substantially aligned with and centered on the vertical axis, and a pin channel in the fixture that is substantially aligned with the horizontal axis. The apparatus also includes a pin that can be removably placed in the pin channel, and a means for depositing a semi-liquid material in the penile cylinder while a tip portion of the penile cylinder is in the cylinder tip cavity of the fixture.

### FIELD OF THE INVENTION

The present invention relates generally to inflatable penile prostheses. The invention relates specifically to an apparatus and method for reinforcement of a penile cylinder.

### BACKGROUND OF THE INVENTION

Inflatable penile prostheses (hereinafter, “IPPs”) are known, and have been successfully used in surgically treating male patients suffering from erectile dysfunction. In some so-called three-piece IPPs, an abdominal reservoir is fluidly connected to (i) a pair of inflatable penile cylinders implanted in a patient's penis and (ii) a pump to transfer fluid between the abdominal reservoir and the penile cylinders.

Typically, an apparatus including a mandrel and a dip tank is employed for manufacturing an IPP cylinder. In such a typical apparatus, the mandrel is in a shape of a cylinder to be created with a distal tip portion and a proximal end portion, and the dip tank contains a semi-liquid material such as that utilized in production of BIOFLEX® brand penile prostheses of Coloplast A/S. The mandrel is dipped, tip portion first, downwardly into the material in the dip tank and then removed from the dip tank after being coated in the semi-liquid material. The coated mandrel is then usually rotated approximately 180 degrees so that the tip portion is oriented upwardly, away from the dip tank so that the semi-liquid material disperses evenly along the mandrel during a curing cycle (also hereinafter referred to as “cured” or “curing”). There may be several dipping and curing cycles needed to adequately “build up” a wall thickness in the cylinder being created on the mandrel, and an average time between such cycles may be of a duration to allow the semi-liquid material to at least partially cure so that successive layers of the material deposited on the mandrel has time to stabilize and “out-gas”. Thus, such dip tank operations can be labor-intensive and time-consuming.

As known to those in the art, a penile cylinder provided to a physician typically includes a hole or other aperture through a tip portion of the cylinder, transverse to a major longitudinal axis of the cylinder, so that a suture may be looped therethrough. The suture may thus be used by the physician during implantation surgery to pull the cylinder into the patient's penis. Therefore, as another step in the manufacturing process of the cylinder, the tip portion may be reinforced by further dipping and curing cycles at the tip portion so that there exists sufficient material thickness to accommodate the hole or aperture through which the suture may be looped as aforementioned. As further steps in the manufacturing process, the hole or aperture may then be provided by a subsequent drilling operation, and the cylinder may be finished in a grinding operation to ensure a uniform outside diameter after creation of the hole or aperture.

Accordingly, there may be a need in the art for an apparatus and method for reinforcement of a penile cylinder of the present invention that might obviate a need for time-consuming and labor-intensive dipping and curing cycles, and finishing processes. Also, there may be a need in the art for an apparatus and method for reinforcement of a penile cylinder of the present invention that might be relatively simple and economical to manufacture.

### SUMMARY OF THE INVENTION

In accordance with basic aspects of the present invention, an apparatus for reinforcement of a penile cylinder includes a fixture having a vertical axis, a horizontal axis, a cylinder tip cavity that is substantially aligned with and centered on the vertical axis, and a pin channel in the fixture that is substantially aligned with the horizontal axis. The apparatus also includes a pin that can be removably placed in the pin channel, and a means for depositing a semi-liquid material in the penile cylinder while a tip portion of the penile cylinder is in the cylinder tip cavity of the fixture.

Also in accordance with basic aspects of the present invention, a method for reinforcement of a penile cylinder includes providing the penile cylinder and an apparatus for reinforcement of the penile cylinder. A tip portion of the penile cylinder can be placed in the cylinder tip cavity of the fixture. The pin can be removably placed in the pin channel of the fixture and through side walls of the tip portion of the penile cylinder. The tip portion of the penile cylinder can be filled with a selected amount of a semi-liquid material, and the semi-liquid material can then be cured. The pin can be removed from the pin channel, and the cylinder can then be removed from the fixture.

### BRIEF DESCRIPTION OF THE DRAWINGS

FIG. 1 is a schematic illustration of an example of an apparatus and method for reinforcement of a penile cylinder of the present invention, depicted before use.

FIG. 2 is a schematic illustration of the example of an apparatus and method for reinforcement of a penile cylinder shown in FIG. 1, depicted in use.

FIG. 3 is a schematic illustration of the example of an apparatus and method for reinforcement of a penile cylinder shown in FIG. 2, depicted in later use.

### DETAILED DESCRIPTION OF THE INVENTION

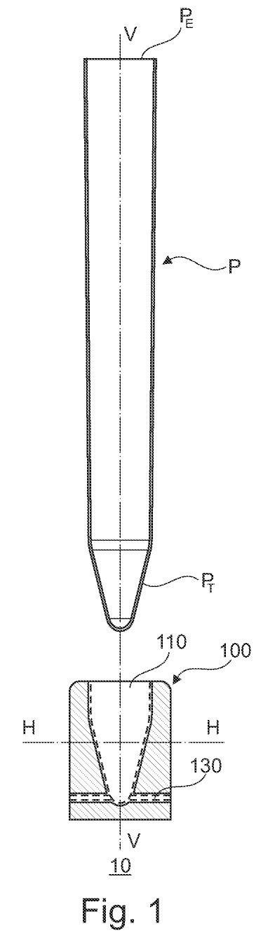

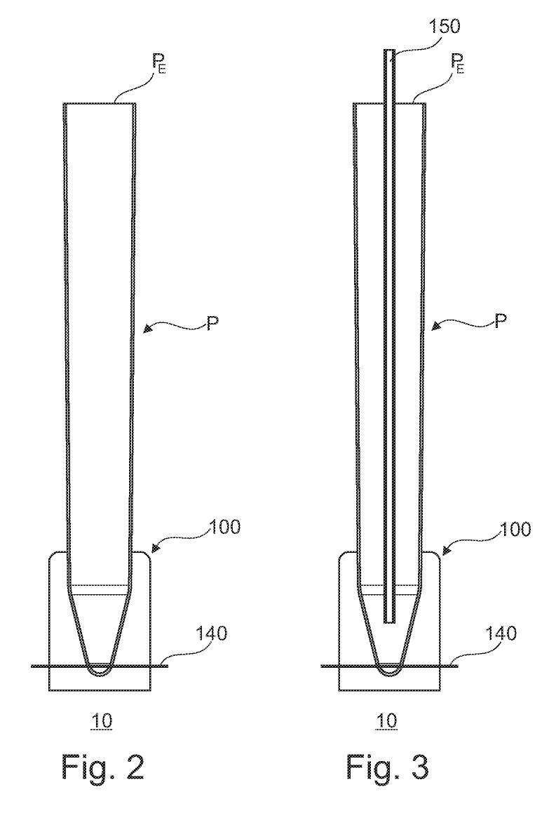

Illustrated in FIGS. 1-3 is an example of an apparatus 10, for reinforcement of a substantially hollow and open penile cylinder P, of the present invention. As will be further described, FIGS. 1-3 also illustrate an example of a method for reinforcement of a penile cylinder. In the following description, the terms “substantially hollow and open” could describe a penile cylinder P that (a) has been partially manufactured with (i) a substantially hollow interior or cavity for containing a fluid for expanding the cylinder in use and (ii) an open end portion P_{E} opposite a closed tip portion P_{T}, but (b) is not yet finished as a complete pressure vessel as known to those of skill in the IPP art. Apparatus 10 could include a fixture 100 defined in part by a vertical axis V and a horizontal axis H, and a cylinder tip cavity 110 that could be substantially aligned with and centered on vertical axis V. Fixture 100 could also include a pin channel 130 in fixture 100 that could be substantially aligned with horizontal axis H. As shown in FIGS. 2-3, apparatus 10 could also include a pin 140 that could be removably placed in pin channel 130. Apparatus 10 could further include a means 150 for depositing a semi-liquid material in substantially hollow and open penile cylinder P while its tip portion P_{T} is in cavity 110 of fixture 100.

Although depicted as a generally rectangular device, fixture 100 could embody any desired geometry. Fixture 100 could also be manufactured in a process including material selected from a group consisting of plastic, steel, and aluminum; and the process to manufacture fixture 100 could be selected from a group consisting of casting, machining, milling, and molding. Furthermore, pin 140 could be manufactured in a process including material selected from a group consisting also of plastic, steel, and aluminum.

It is to be understood that, although depicted in FIGS. 1-3 as extending fully through fixture 100, pin channel 130 could, alternatively for example, extend through one side of cavity 110 and only as far as minimally into an opposite side of cavity 110 so that pin 140 may be just minimally supported when inserted therewithin.

For use with a typical penile cylinder P, fixture 100 could have, for example, solid rectangular dimensions of about 3.175 to 4.445 cm (1.25 to 1.75 inches) in height and 2.540 to 5.080 cm (1.00 to 2.00 inches) in width and depth. Correspondingly, cavity 110 could have a circular diameter of about 1.466 to 1.905 cm (0.577 to 0.750 inches) at an exterior surface of fixture 100, tapering to about dimensions of a tip of a penile cylinder P within fixture 100 near channel 130. Channel 130 and pin 140 could, also correspondingly, have a diameter of about 0.508 to 0.813 cm (0.200 to 0.320 inches)

It is to be appreciated and understood that a particular embodiment of apparatus 10 could be produced using any suitable materials and fabrication techniques, depending upon particular requirements and configurations of each component of a particular apparatus 10. As such, suitable materials and fabrication techniques could, individually or in combination, employ several different materials (e.g., the aforementioned plastic, steel, and aluminum, etc.) and several different fabrication steps (e.g., the aforementioned casting, machining, milling, and molding, etc.).

With reference now to FIGS. 1-3, an example of a method for reinforcement of a penile cylinder of the present invention could include the following steps.

A substantially hollow and open penile cylinder P could be provided, along with apparatus 10. Tip portion P_{T} of cylinder P could be placed in cylinder tip cavity 110 of fixture 100. Pin 140 could then be removably placed in pin channel 130 of fixture 100 and through side walls of tip portion P_{T} of cylinder P. In this regard, as would be understood by those in the IPP art and as shown in FIGS. 1-3, cylinder P could be of a generally hollow cylindrical configuration with a generally conical portion including tip portion P_{T} that is closed at its tip. Thus, cylinder P could be characterized as having side walls through which pin 140 could pierce substantially along horizontal axis H adjacent to channel 130 of fixture 100 when cylinder P is in fixture 100. Then, means 150 could fill tip portion P_{T} with a selected amount of a semi-liquid material to cover pin 140 therewithin. The semi-liquid material could be selected from a group consisting of a medical grade thermoplastic material, a medical grade thermoset material, and a medical grade polymer-like material. The semi-liquid material could be further selected from a group consisting of polyurethane and silicone. The semi-liquid material could be cured and pin 140 could then be removed from channel 130 in fixture 100, leaving a hole or aperture as desired through tip portion P_{T} of cylinder P. Finally, cylinder P could be removed from fixture 100, and finished for use as an IPP component.

Although not depicted in the drawings, it is to be appreciated and understood that tremendous efficiency could possibly be attained by employing an “assembly line” of several fixtures 100 for, correspondingly, simultaneous creation of holes or apertures in several cylinders P.

It is to be appreciated, from the foregoing disclosure that the present invention might provide heretofore unattainable efficiency in obviating a need for time-consuming and labor-intensive dipping and curing cycles, and finishing processes. Furthermore, it is to be appreciated from the foregoing that the present invention uniquely and advantageously satisfies a long-felt need for an apparatus and method for reinforcement of a penile cylinder that might be relatively simple and economical to manufacture.

It will be appreciated by those in art of IPP manufacturing that novel aspects of the apparatus and method of the present invention could be capable of use in, and beneficial to, virtually any dipping processes per se—even outside of surgically implantable medical technologies.

While the present invention has been particularly shown and described with reference to the accompanying specification and drawings, it will be understood however that other modifications thereto are of course possible; and all of which are intended to be within the true spirit and scope of the present invention. It should be appreciated that (i) components, dimensions, shapes, and other particulars of example embodiments of the invention aforedescribed may be substituted for others that are suitable for achieving desired results, (ii) various additions or deletions may be made thereto, and (iii) features of the foregoing examples may also be made in combinations thereof. It is also to be understood in general that any suitable alternatives may be employed to provide the apparatus and method for reinforcement of a penile cylinder of the present invention.

Lastly, of course, the choice of compositions, sizes, and strengths of various aforementioned elements of the present invention are all a matter of design choice depending upon intended uses thereof. Accordingly, these and other various changes or modifications in form and detail of the present invention may also be made therein, again without departing from the true spirit and scope of the invention as defined by the appended claims.

What is claimed is:

1. An apparatus for reinforcement of a penile cylinder, comprising:
a fixture having (i) a vertical axis, (ii) a horizontal axis, (iii) a cylinder tip cavity substantially aligned with and centered on said vertical axis, and (iv) a pin channel in said fixture and substantially aligned with said horizontal axis;
a pin, removably placed in said pin channel; and
a means for depositing a semi-liquid material in the penile cylinder while a tip portion of the penile cylinder is in said cylinder tip cavity of said fixture.

2. The apparatus for reinforcement of a penile cylinder of claim 1, wherein said fixture is manufactured in a process including material selected from a group consisting of plastic, steel, and aluminum.

3. The apparatus for reinforcement of a penile cylinder of claim 2, wherein said process includes a technique selected from a group consisting of casting, machining, milling, and molding.

4. The apparatus for reinforcement of a penile cylinder of claim 1, wherein said pin is manufactured in a process including material selected from a group consisting of plastic, steel, and aluminum.

5. A method for reinforcement of a penile cylinder, comprising steps of:
providing the penile cylinder;
providing an apparatus for reinforcement of a penile cylinder of claim 1;
placing a tip portion of the penile cylinder in said cylinder tip cavity of said fixture;
removably placing said pin in said pin channel of said fixture and through side walls of the tip portion of the penile cylinder;
filling said tip portion of the penile cylinder with a selected amount of a semi-liquid material;
curing said semi-liquid material;
removing said pin from said pin channel; and
removing the cylinder from said fixture.

6. The method for reinforcement of a penile cylinder of claim 5, wherein said semi-liquid material is selected from a group consisting of a medical grade thermoplastic material, a medical grade thermoset material, and a medical grade polymer-like material.

7. The method for reinforcement of a penile cylinder of claim 5, wherein said semi-liquid material is further selected from a group consisting of polyurethane and silicone.
